(19) United States
(12) Patent Application Publication
(10) Pub. No.: US 2007/0042491 A1
(43) Pub. Date: Feb. 22, 2007

(54) Amplification of cell populations from embryonic stem cells

(76) Inventors: Jeffrey M. Karp, Brookline, MA (US); Lino da Silva Ferreira, Cambridge, MA (US); Ali Khademhosseini, Cambridge, MA (US); Robert S. Langer, Newton, MA (US)

Correspondence Address: CHOATE, HALL & STEWART LLP, TWO INTERNATIONAL PLACE, BOSTON, MA, 02110, US

(21) Appl. No.: 11/505,683

(22) Filed: Aug. 17, 2006

Related U.S. Application Data
(60) Provisional application No. 60/709,467, filed on Aug. 18, 2005.
(60) Provisional application No. 60/712,466, filed on Aug. 29, 2005.

(51) Int. Cl. C12N 5/08

(52) U.S. Cl. 435/366; 435/368; 435/370

(57) ABSTRACT

Culturing embryonic stem cells without the use of embryoid bodies leads to a increase in the frequency of predetermined cell types.

This application claims priority to U.S. Provisional Application No. 60/709,467, filed Aug. 18, 2005, and U.S. Provisional Application No. 60/712,466, filed Aug. 29, 2005, the entire contents of both of which are incorporated herein by reference.

### FIELD OF THE INVENTION

This invention relates to culture techniques for promoting the differentiation of embryonic stem cells.

### BACKGROUND OF THE INVENTION

Currently, there is an overwhelming shortage of donor organs, tissues and cells for the repair of traumatic tissue injury or deficiency arising from genetic conditions and tumors, and for treating age related diseases, e.g., osteodegenerative diseases such as osteoporosis and osteoarthritis. Current therapies for tissue defects, including autograft and allograft transplantations, have inherent limitations such as donor site morbidity and host immune rejection. Alternative therapies being considered involve the combination of liquid, gel, or solid carriers with a source of cells. In one modality, progenitor cell numbers are expanded in vitro, placed onto biodegradable scaffolds in combination with factors that stimulate differentiation, e.g., osteogenic differentiation, followed by implantation into a defect site [1, 2]. Ideally new tissue will grow, the scaffold will degrade, and the patient will be left with functional tissue. In choosing an appropriate cell source for these types of tissue engineering strategies, one must consider the capacity of the chosen cells to differentiate into cells which can produce the appropriate extracellular matrix.

During the past two decades there has been significant progress in our understanding of stem cells which may be defined by their capacity for self-renewal and multilineage differentiation. Although there has been great interest in locating and expanding adult stem cells [3], this approach is restricted by isolation difficulties and limited quantities [4]. An alternative cell source consists of embryonic stem cells (ESC) which are pluripotent cells derived from the inner cell mass of embryos at the blastocyst stage [5]. Embryonic stem cells (ESC) offer a potentially unlimited supply of cells that may be driven down specific lineages to give rise to all cell types in the body [3-5]. Although cells derived from the mouse can be used as a tool to better understand the process of differentiation, there are considerable differences between murine and human embryonic stem cells (hESC) [14]. Therefore, understanding cues that induce specific differentiation of hESC is important for tissue engineering and regenerative medicine.

Two methods have been examined in an attempt to stimulate the differentiation of hESC into osteogenic cells. In one method, osteogenic cells are derived from 3-dimensional cell spheroids called embryoid bodies (EB) [11, 12]. EB can be formed from either single cell suspensions of ESC or from aggregates of cells. EB mimic the structure of the developing embryo and recapitulate many of the stages involved during its differentiation [15], and clonally derived EB can be used to locate and isolate tissue specific progenitors. An alternative system that has been tested but not well characterized avoids EB through the immediate separation of ESC colonies into single cells which are then plated directly into a cell adhesive culture dish [9, 11]. However, EB were avoided because the cells did not readily form EB. Stains for calcium and phosphorus (e.g., Alizarin Red and von Kossa) [12]) did not localize to the same regions [11] in mineralized ECM produced by hESC derived with and without an embryoid body step, which may indicate dystrophic mineralization.

### SUMMARY OF THE INVENTION

In one aspect, the invention is a method of culturing embryonic stem cells. The method includes providing a plurality of embryonic stem cells (ESC), seeding the ESC on a substrate or suspending them in a culture medium, and incubating the seeded or suspended ESC under conditions in which they can differentiate to obtain a population of stem cells. The ESC are not stimulated to form embryoid bodies, and the proportion of a predetermined differentiated cell type in the population of cells is at least two times greater than the proportion of the predetermined differentiated cell type in the population obtained in the same manner except that the ESC are simulated to form embryoid bodies. For example, the proportion may be at least three times greater, at least four times greater, at least five times greater, at least six times greater, or at least seven times greater. The method may further include culturing a population of ESC over a feeder layer. The cultured cells may be separated into individual cells, or aggregates of cells may form within the population, which aggregates may be seeded. Cells may be seeded on a two dimensional or a three dimensional substrate. Suspending may include encapsulating the ESC in a hydrogel and placing the encapsulated ESC in a culture medium. The ESC may be human ESC. The method may further include culturing the seeded or suspended cells in the presence of at least one growth factor or cytokine.

In another aspect, the invention is a method of obtaining a population of osteogenic cells. The method includes providing a plurality of ESC, seeding the ESC on a substrate, and incubating the seeded ESC in the presence of ascorbic acid, dexamethasone, and beta-glycerophospate. The ESC are not stimulated to form embryoid bodies. The proportion of osteogenic cells in the incubated ESC may be at least two times greater than the proportion of osteogenic cells in a population of cells obtained in the same manner, except that the ESC are stimulated to form embryoid bodies. The seeded ESC may produce bone nodules during the step of incubating, and the elapsed time before the bone nodules are produced may be less than, for example, at least 20%, at least 30%, at least 40%, at least 50%, or at least 60% less than an elapsed time for a population of cells obtained in the same manner except that the ESC are stimulated to form embryoid bodies.

### BRIEF DESCRIPTION OF THE DRAWING

The invention is described with reference to the several figures of the drawing, in which,

FIG. 1: A comparison of culture protocols.

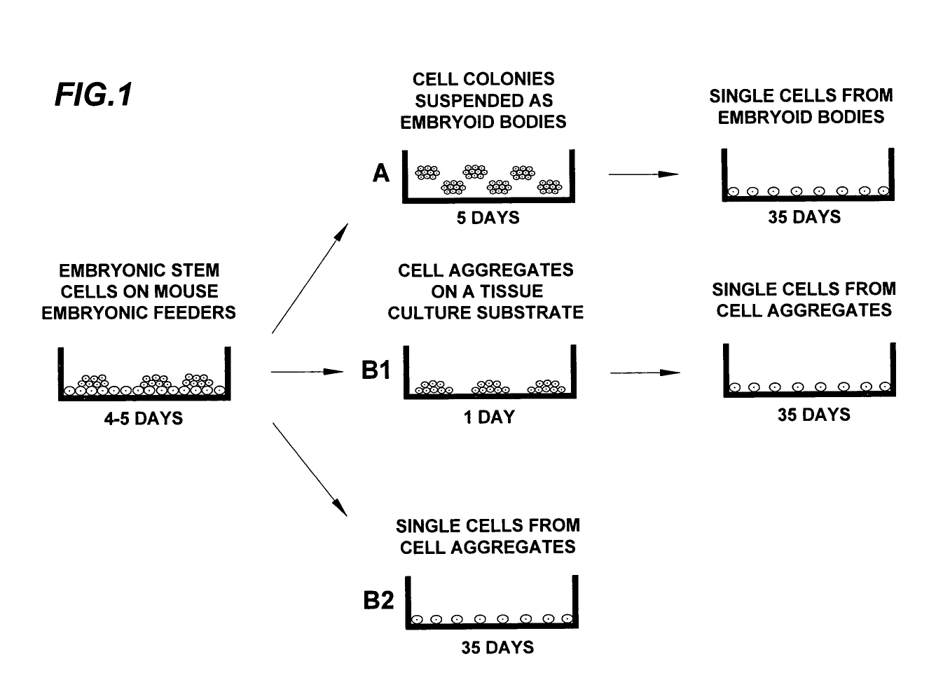

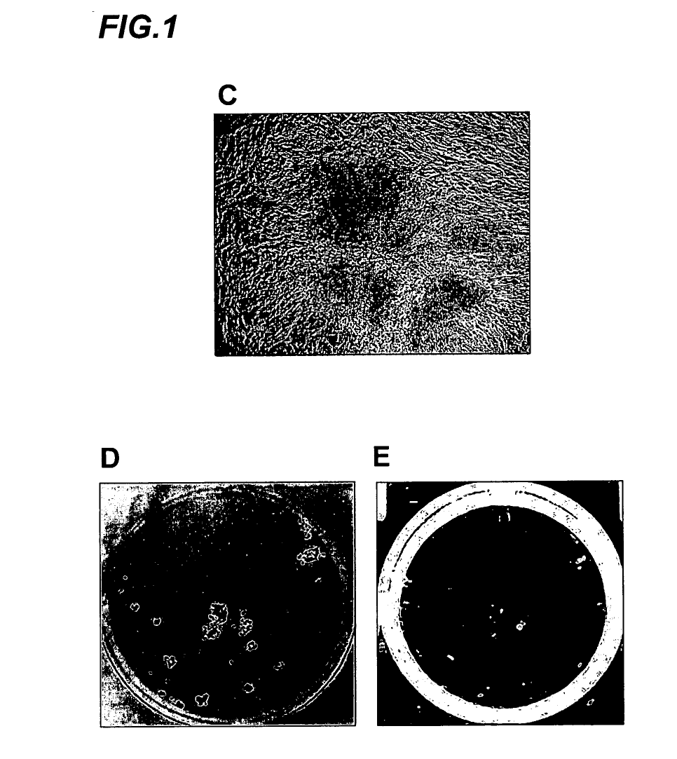

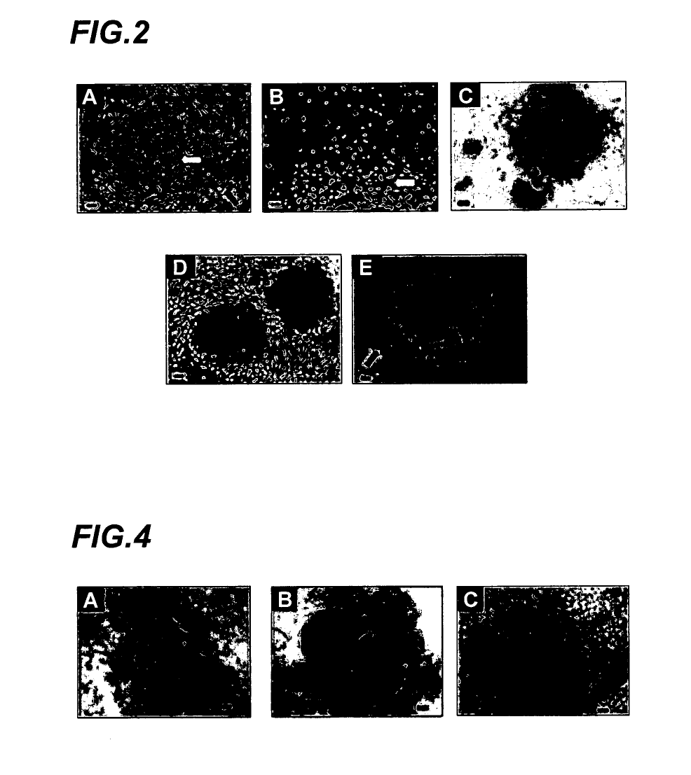

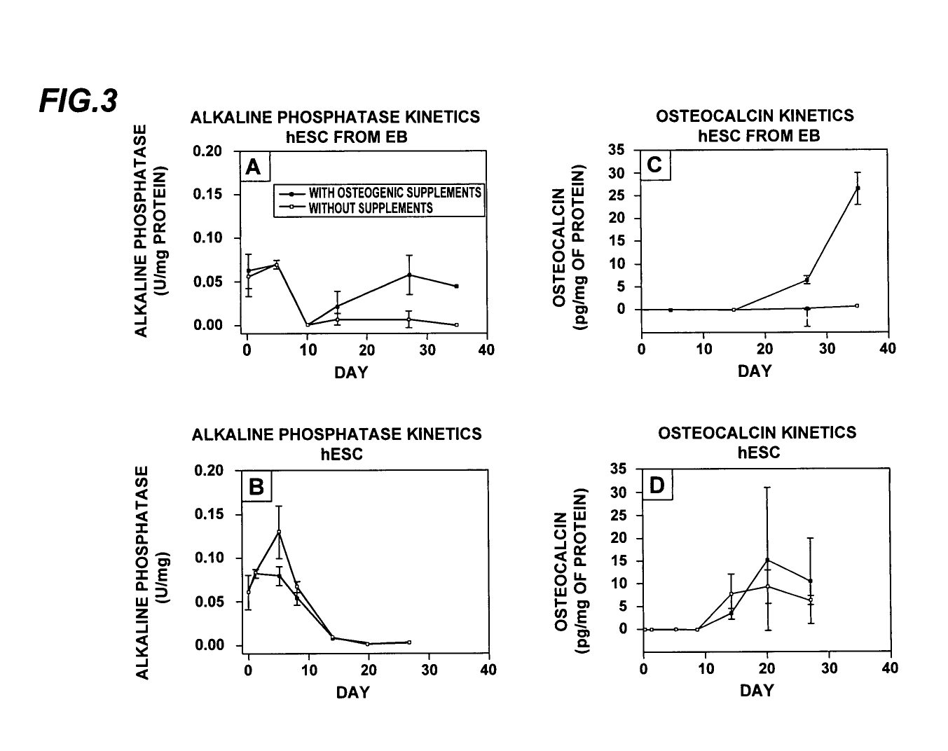

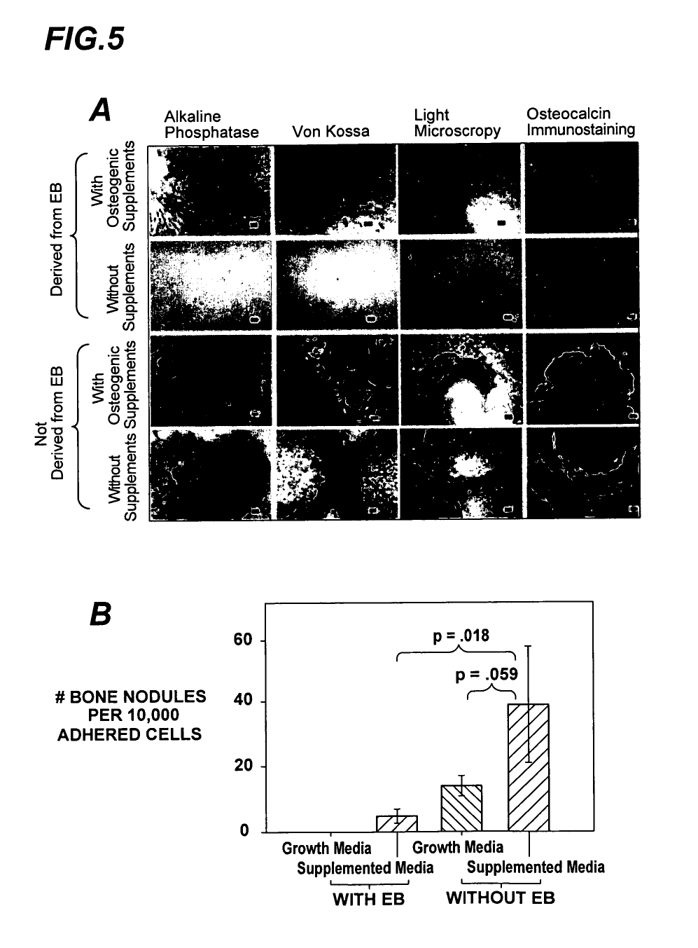

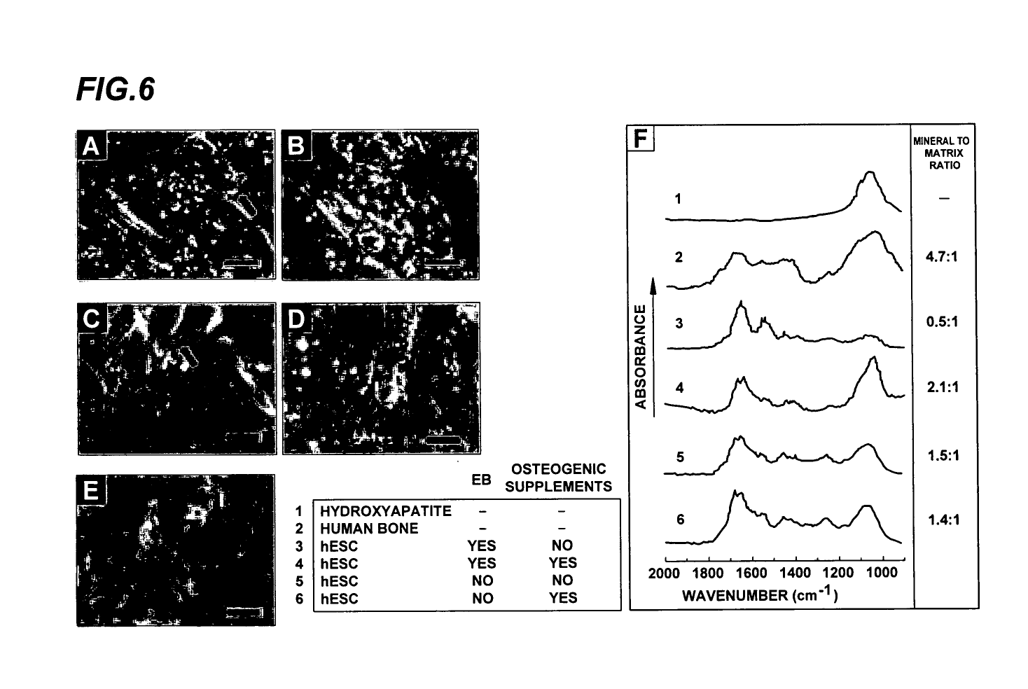

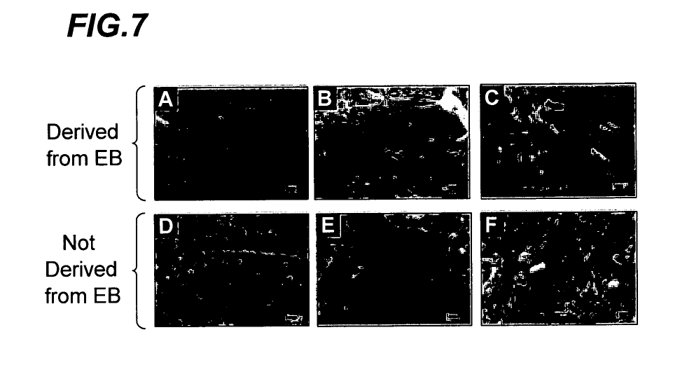

hESC were cultured on mouse embryonic fibroblast feeder layers for 4 days. The cell aggregates were removed from the mouse feeders with collagenase IV and (A) suspended as EB for 5 days and then plated as a single cell suspension, (B-1) directly placed onto tissue culture petri dishes for 1 day followed by plating as a single cell suspension according to an exemplary embodiment, or (B-2) directly plated as a single cell suspension without the 1 day plating of cell aggregates. Cultures from all protocols were grown for up to 35 days. After 12-15 days in culture, bone nodules according to protocol B-1 were observed by light microscopy (FIG. 1C), von Kossa (FIG. 1D) and tetracycline (FIG. 1E). Similar bone nodules were observed after 12-15 days using protocol B-2, and after 28 days according to protocol A.

FIG. 2: Examination of the cell colonies during various stages of culture.

(A) Light microscopic images of hESC colonies (white arrow) grown on mouse feeders (black arrow). (B) The primitive state of the hESC aggregates (white arrow) prior to differentiation experiments was verified by positive staining for the rhodamine-conjugated ESC marker OCT-4 (red). The mouse feeders (background) only stained for the blue 4′, 6-diamidino-2-phenylindole (DAPI) nuclear stain. (C) Day 5 hESC aggregates stained for both ALP and von Kossa were positive for ALP, unlike surrounding mouse feeders. No evidence for von Kossa staining was observed indicating that the aggregates did not contain any mineralized regions. (D) hESC aggregates were removed from the mouse feeders by treatment with colagenase IV and then (E) placed onto a tissue culture dish for 24 hours according to an exemplary embodiment. During this time, cells migrated from the aggregate across the culture substrate and displayed a flattened morphology as observed with (E) light microscopy. Scale bars: 100 μm (A-D), 200 μm (E).

FIG. 3: Kinetics of ALP and osteocalcin (OCN) expression

(A) Undifferentiated hESC initially displayed high levels of alkaline phosphatase (ALP) that remained unchanged during the 5 day suspension as EB. The ALP signal decreased sharply to undetectable levels after culturing single cells, derived from the EB, in both media with and without osteogenic supplements. Following this, a sizeable increase in ALP signal was only observed with cells cultured in the presence of L-ascorbic acid (AA), sodium β-glycerophosphate (βgP) and dexamethasone (DEX). When EB were omitted from the culture protocol, (B) a 35% increase (p=0.143, n=3) in ALP was observed while culturing the cells for 24 h as cell aggregates in the absence of mouse feeders according to an exemplary embodiment. This was followed by a 58% increase (p=0.052, n=3) within cultures without supplements followed by a gradual decrease for both groups until reaching a plateau slightly above zero. For cells derived from EB, (C) OCN signal was only detected when osteogenic supplements were used and showed an upward trend when the cultures were terminated. (D) For cells derived without EB according to an exemplary embodiment, OCN was detected earlier and quickly reached a plateau regardless of the presence of osteogenic supplements.

FIG. 4: Culturing hESC without EB leads to the formation of three dimensional structures with mineralized areas containing calcium phosphate

(A) After 10-12 days, the matrix produced by hESC cultured without EB according to an exemplary embodiment, in both the presence and absence of supplements, stained for both ALP and von Kossa. (B) After 12 days, 3D hemispherical structures emerged displacing many of the mineralized regions to the margins of these structures (black arrow), which was followed by a substantial decrease in ALP signal (not shown). (C) The mineralized matrix did not stain for safranin-O indicating that these regions did not contain glycosaminoglycans which are associated with cartilage. All scale bars represent 1 μm.

FIG. 5: The hESC culture protocol affects both the gross morphology and the quantity of bone nodules.

(A) ALP, von Kossa, and OCN immunocytochemical staining of hESC after 35 days in culture. Cells derived from EB only showed significant ALP, von Kossa, and OCN staining when cultured in the presence of osteogenic supplements compared to the condition without supplements. In contrast, cells derived without EB according to an exemplary embodiment stained for ALP, von Kossa, and OCN irrespective of addition of osteogenic supplements. Regardless of the inclusion of EB, OCN and tetracycline (not shown) staining localized to similar regions that stained positive for von Kossa. Scale bars: 100 μm for ALP and von Kossa stained images, and 500 μm for Immunocytochemistry images. (B) Frequency of bone nodule formation per 10,000 adhered cells. Cells grown from EB produced bone nodules only in the presence of osteogenic supplements (5.1±2.4). In comparison, cells grown without EB according to an exemplary embodiment cultured in growth media and media containing osteogenic supplements produced 13.8±3.1 and 39.1±17.8 bone nodules per 10,000 plated cells, respectively.

FIG. 6: hESC are capable of producing morphologically identifiable bone that contains calcium and phosphorus with an apatitic phase.

SEM was used to examine the morphology of the matrix produced by osteogenic cultures of hESC. Regardless of whether cells were derived from EB (A, B) or not (C, D), cultures produced thin collagenous fibers which appeared to display traces of ectopic mineral as observed by their grainy appearance (arrow) even in the absence of supplements (A,C). The collagenous matrix became mineralized (B, D) in the presence of AA, βgP and DEX. In comparison, (E) the matrix within human bone contained thick, densely packed collagen fibers. (F) FTIR spectra and mineral to matrix ratios (obtained by integrating the area under the curve between 900-1200 cm⁻¹ (phosphate bands) and dividing by the area under the curve between 1585 and 1725 cm⁻¹ (amide I band)). The mineral peak from hydroxyapatite and the mineral and matrix peaks from human bone were comparable to the extracellular matrix produced by the hESC irrespective of whether the cells were derived from EB. However, hESC cultures derived from EB in the absence of supplements produced a substantially smaller mineral peak which corresponded to a low calcium to phosphate ratio. Scale Bars: 10 μm (A-E).

FIG. 7: Examination of the bone matrix/tissue culture interface with SEM

(A-E) The matrix/culture dish interface was revealed through removal of cells and extracellular matrix. (A,D) cultures in the absence of, and (B, C, E, F) in the presence of osteogenic supplements. Unlike (A) cells grown in the absence of supplements, (B,C) cultures derived from EB grown in the presence of osteogenic supplements displayed 1 μm sized mineralized globular accretions (black arrow), which were reminiscent of the cement line formed by differentiating osteogenic cells. These globules were produced by the cells through pseudopodia (grey arrow). In contrast, (D, E) cement line globules were not observed at the matrix/culture dish interface when hESC were obtained without EB according to an exemplary embodiment, regardless of the supplementation regime. However, when these cells were cultured in the presence of osteogenic supplements, (F) 1-μm sized globules (black arrows) were observed entrapped within the overlying collagenous matrix (white arrow). All scale bars represent 1 μm.

### DETAILED DESCRIPTION OF CERTAIN PREFERRED EMBODIMENTS

Because each ESC has the capacity to differentiate into multiple cell types and that the formation of EB's leads to the formation of numerous cell types, we hypothesized that, by omitting the EB step, one could provide a more uniform cell microenvironment and direct the differentiation of the ESC more homogeneously. In one embodiment, osteogenic cells derived from hESC can produce many of the morphological hallmarks of de novo bone formation. The cells may be directed towards a specific cell type or phenotype. The cells need not be directed all the way towards a fully differentiated phenotype but may be differentiated into various types of progenitor cells at various stages of differentiation, e.g., mesoderm or osteogenic.

Prior art methods for promoting the differentiation of embryonic stem cells (ESC) often involve culturing these cells in embryoid bodies (EBs) for some amount of time. The EB are either plated for further culture or trypsinized to obtain a suspension of dissociated cells. Cao, et al., report culturing EB themselves under conditions that promote osteogenic lineages, without first dissociating the EB (Tissue and Cell, (2005) 37:325-334). The EB stage recapitulates the development of ESC into multiple tissues simultaneously during embryonic development. Furthermore, the differentiation of ES is influenced by a large number of factors, including cell-cell interactions and the immediate cell environment. In the three-dimensional ball of cells that is the embryoid body, each cell essentially has a unique environment. Cells in the interior of the EB receive nutrients from culture media that have essentially been filtered through the cells in the exterior portions of the EB, and those exterior cells may produce factors and even waste products that influence the development of interior cells. Thus, culture of the EB results in a large number of different cell types from which it may be difficult to isolate particular cells.

In one embodiment, the invention provides a method of culturing ESC that promotes the amplification of specific cell populations with respect to other cell types that may appear in the same culture. In general, ESC, for example, mouse or human ESC, are cultured without the use of EB. Undifferentiated cells may be grown on feeder layers or in the presence of factors such as bFGF to expand the cells in an undifferentiated state before being transferred to a culture in which they will differentiate. Cells incubated on feeder layers tend to develop into aggregates of cells. These aggregates may be separated into single cells before being transferred to a culture in which they will differentiate. Alternatively, aggregates may be separated into smaller clumps of cells before transfer. In some embodiments, to increase the number of adherent cells, cell aggregates may first be plated onto a substrate, after which the cells are separated into smaller clumps or groups or into a single cell suspension. In some embodiments, undifferentiated cells that have not been plated or otherwise cultured in an environment where they may begin to spontaneously differentiate, may be separated from one another and used as single cells. Undifferentiated cells may be distinguished by expression of OCT-4, alkaline phosphatase (ALP), and nanog (Donovan P J., Nat Genet 2001; 29(3):246-7; Niwa H, et al, Nat Genet 2000; 24(4):372-6; Aubin J E, et al., Bone 1995; 17(2 Suppl):77S-83S). Undiffereniated hESC may be further distinguished by an absence of SSEA-1 expression. Undifferentiated hESC may also express of TRA-1-60 and/or TRA-1-81. In some embodiments, at least 90%, at least 95%, or at least 99% of hESC express one or more of OCT-4, ALP, nanog, TRA-1-60, and TRA-1-81 and do not express SSEA-1. Undifferentiated murine ESC may be further distinguished by expression of SSEA-1 but do not express SSEA-3 or SSEA-4, which may be expressed during differentiation. In some embodiments, at least at least 90%, at least 95%, or at least 99% of the mESC express one or more of OCT-4, ALP, nanog, and SSEA-1 and do not express SSEA-3 or SSEA-4.

Single cells may be plated or seeded directly onto a substrate or first combined with a liquid, gel, or solid carrier, as described below. Differentiation along specific paths may be induced or enhanced through the use of growth factors, physical stimulation, or through contact with other cell types or particular substrates. The techniques described herein may be applied to any ESC, for example, mouse ESC or human ESC. These methods allow the ESC to experience more homogeneous cues for differentiation. The proportion of a particular differentiated cell in the culture may be at least 2, at least 3, at least 4, at least 5, at least 6, or at least 7 times greater than the proportion of the cell in a culture prepared using EB. The differentiated cells may be fully differentiated cells or multipotent precursor cells that are more differentiated than the original ESC but not completely differentiated. In some embodiments, the omission of an EB stage can speed differentiation by at least 20%, at least 30%, at least 40%, at least 50%, or at least 60%. One skilled in the art will recognize that the acceleration and efficiency of differentiation will depend on factors such as the desired differentiation path, the particular growth factors used, and the culture conditions, e.g., the use and type of a substrate or bioreactor.

Carriers

Single cells may be combined with a carrier before being seeded on a substrate. For example, they may be combined with Matrigel or Growth Factor Reduced Matrigel, available from Becton-Dickinson. Unmodified Matrigel is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Growth Factor-Reduced Matrigel is produced by removing most of the growth factors from the matrix (see Taub, et al., Proc. Natl. Acad. Sci. USA, (1990);87(10):4002-6). Additional gels that may be employed as carriers include but are not limited to those formed from—alginate, fibrin, agar, dextran, chitosan, hyaluronic acid, and/or any of collagen types I-IX. Peptide hydrogels, whether self-assembled or not, may also be employed. Synthetic hydrogel materials polymerized from such monomer/macromer mixtures made of 2-hydroxyethylmethacrylate (HEMA), methyl methacylate (MMA), N-vinyl pyrorolidone (NVP), methacrylic acid (MA), vinyl alcohol (VA), tris-(trimethylsiloxysilyl), propylvinyl carbamate (TPVC), dimethysiloxy di [silylbutanol] bis[vinyl carbamate] (PBVC), polyethylene glycol (PEG), trimethylsiloxy silane (TRIS), siloxane macromers; synthetic rubbery or plastic polymers such as poly(epsilon-caprolactone) (PCL) and poly (D,L-lactic-co-glycolic acid) (PLGA) are also appropriate for use with embodiments of the invention.

Where a carrier is used, it may also include other extracellular matrix components, such as glycosaminoglycans, fibrin, fibronectin, proteoglycans, and glycoproteins. The carrier may also include basement membrane components such as collagen IV and laminin. In one embodiment, extracellular matrix components found in tissues containing the same type of cells as the stem cells are being differentiated into may be incorporated into the gels. Enzymes such as proteinases and collagenases may be added to the gel, as may cell response modifiers such as growth factors, cytokines, and chemotactic agents.

Substrates

Single cells, whether combined with a carrier or not, may be cultured in a culture medium suspension, on conventional two dimensional plates or on three dimensional scaffolds. Two-dimensional plates may be coated with materials that are more conducive to a particular path for cell differentiation. Three-dimensional scaffolds may include hydrogels or more fibrous polymers. Indeed, hydrogels and more fibrous polymers may be combined to make composite scaffolds in which pores in three-dimensional polymer scaffolds are filled with the hydrogel. Alternatively or in addition, cells suspended in Matrigel or Growth Factor Reduced Matrigel may be deposited on three-dimensional scaffolds, and the Matrigel allowed to run into the pores. Exemplary methods of choosing appropriate polymers are described in U.S. Patent Publication No. 20050019747, published Jan. 27, 2005, the contents of which are incorporated herein by reference.

A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference). Other exemplary polymers for forming either two dimensional or three dimensional scaffolds include PLA, PGA, PLGA, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes, polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of any of the above, adducts of any of the above, and mixtures of any of the above polymers, co-polymers, and adducts with one another

Non-polymer materials, for example, ceramic, metal, or composite materials including two or more of metals, ceramics, or polymers, may also be employed as both two and three-dimensional substrates. Exemplary materials include alumina, calcium carbonate, calcium sulfate, calcium phosphosilicate, sodium phosphate, calcium aluminate, calcium phosphate, hydroxyapatite, α-tricalcium phosphate, dicalcium phosphate, β-tricalcium phosphate, tetracalcium phosphate, amorphous calcium phosphate, octacalcium phosphate, silicates, and biocompatible metals and alloys such as cobalt-chromium alloys, Ti-6Al-4V, commercially pure titanium, zirconium and its biocompatible alloys, niobium, and biocompatible steels. Methods of making three-dimensional substrates with these materials include those disclosed in U.S. Pat. No. 6,530,958, the contents of which are incorporated herein by reference.

Substrates may be textured to promote differentiation along a particular lineage. For example, grooved substrates will promote cellular elongation, a characteristic of nerve and muscle cells. Even two dimensional substrates may be given a fibrous texture to facilitate cell-substrate interactions that resemble interactions with extracellular matrix. Bumps, depressions, striations, cross-hatching at various angles, and other textures may promote one differentiation path with respect to another. Three dimensional polymer substrates may also be textured. For example, polymer scaffolds may be produced with fibers oriented generally along one direction. Alternatively or in addition, the pore size of three dimensional substrates may be adapted to resemble that of a particular tissue or to favor development of a certain size cell.

Cells may be encapsulated in gels or other materials and suspended in media instead of being cultured on plates. For example, cells may be encapsulated in alginate or other hydrogel, for example, using the techniques described in U.S. Pat. No. 4,391,909 by Lim, issued Jul. 5, 1983, the contents of which are incorporated herein by reference. Briefly, a suspension of cells and the gel forming material is forced through a vibrating capillary tube placed within the center of the vortex created by rapidly stirring a solution of a multivalent cation. Droplets ejected from the tip of the capillary immediately contact the solution and gel as spheroidal shaped bodies. A permanent semipermeable membrane may be formed about the capsules by cross-linking surface layers of a hydrogel of the type having free acid groups with polymers containing acid reactive groups such as amine or imine groups, for example, polyethylenimine and polylysine. This is typically done in a dilute solution of the selected polymer. Permanent crosslinks are produced as a consequence of salt formation between the acid reactive groups of the crosslinking polymer and the acid groups of the polysaccharide gum. Nutrients and other factors in the media will diffuse into the capsules, and wastes produced by the cells will diffuse out. Materials produced by the cells may also diffuse out of the capsules, providing a method of characterizing cellular activity without physically disturbing the cells. The capsules may be suspended in practically any volume of media, for example, in large bioreactors, in the absence of a cell adherent substrate, allowing large numbers of cells to be cultured while still being seeded on a polymer support. Alternatively or in addition, single cells may be cultured in bioreactors. Exemplary bioreactors may include a flask or other container that contains media agitated by methods such as but not limited to magnetized stirrers, fluidized bed techniques, or injection of an appropriate gas, the bubbles of which will agitate the media. An exemplary bioreactor is described in U.S. Patent Publication No. 20040137612, published Jul. 15, 2004, the entire contents of which are incorporated herein by reference. For a cell population having a certain frequency of a particular lineage, the use of techniques that may be used to culture large numbers of cells will increase the number of cells of a particular type.

In some embodiments, microfluidic techniques may be used to provide both temporal and physical control over the cellular environment. For example, ESC seeded on a substrate patterned with fluid channels may be exposed to certain growth factors or cytokines at specific points in time. Polymers or extracellular matrix materials may be patterned on a substrate to provide precise control, for each cell, of the surface chemistry with which it interacts. Alternatively, substrates may be prepared that will expose the ESC to an anisotropic environment, for example, with different substrate materials on each side of the cell. Exemplary microfluidic methods for culturing cells are described in U.S. Patent Publications Nos. 20030215941, published Nov. 20, 2003, 20040106192, published Jun. 3, 2004, and 20020173033, published Nov. 21, 2002, and in Chung, et al., 2005, Human neural stem cell growth and differentiation in a gradient-generating microfluidic device. Lab Chip 5:401-406, the contents of which are incorporated herein by reference.

Physical Stimuli

The mechanical interactions of cells and their extracellular matrix influence cellular processes. To further promote differentiation along a desired path, exogenous mechanical forces may be used as a cell response modifier to mimic the mechanical forces exerted by tissues. For example, endothelial cells are exposed to shear forces as blood flows through arteries and veins. Muscle, because it is anchored to bones at least at its ends, is exposed to both uniform and non-uniform tensile stresses. Bone is subjected to compressive and bending stresses during normal locomotion. Organ tissues are exposed to hydrostatic stresses and other compressive stresses. Imposition of mechanical forces on cell-seeded matrices in vitro will influence the production of actin by the seeded stem cells, in turn influencing the degree and type of metabolic activity of the cells and the microstructure of the extracellular matrix they produce. In some embodiments, it may be desirable to flow media over the ESC to create a shear force on a culture. The flow may be pulsed to resemble the flow of fluid through the vascular system. Alternatively or in addition, ESC may be grown on flexible substrates, and the substrates may be stretched in a particular direction, either constantly or with some frequency, to promote elongation and/or alignment.

Similarly, electrical stimulation may be used to influence cell differentiation and metabolism. For example, bone is piezoelectric, and muscle contracts and relaxes in response to electrical signals conducted through nerves. In vitro electrical stimulation imitating the electrical activity of the desired tissue may cause ES cells seeded on a three-dimensional scaffold to produce tissue having the electrical characteristics of that tissue. The use of electrical stimuli to promote tissue development by myoblasts is discussed in U.S Patent Publication 20050112759, published May 26, 2005, the contents of which are incorporated herein by reference.

The shape and microstructure of the polymer matrix and the exogenous forces imposed on the seeded polymer may be optimized for a specific tissue. For example, a medium may be circulated through a seeded tubular substrate in a pulsatile manner (i.e., a hoop stress) to simulate the forces imposed on an artery, or the medium may be used to exert a shear stress on stem cells lining the inside of a tube (Niklason, et al., (1999) Science 284, 489-93; Kaushall, et al., (2001) Nat. Med., 7, 1035-1040). The polymer strands in a three dimensional substrate may be aligned to mimic the tissue structure of muscle, bone, tendon, ligament, or other tissues or formed into tubular networks to promote the formation of vasculature.

Biological Stimuli

In an exemplary embodiment, a cell response modifier such as a growth factor or a chemotactic agent may be added to the ESC culture to promote differentiation along a particular path. Exemplary cytokines and growth factors that may be exploited for use with the invention include but are not limited to dexamethasone, leptin, sortilin, transglutaminase, prostaglandin E, 1,25-dihydroxyvitamin D3, ascorbic acid, β-glycerol phosphate, TAK-778, statins, interleukins such as IL-3 and IL-6, growth hormone, steel factor (SF), activin A (ACT), retinoic acid (RA), epidermal growth factor, bone morphogenetic proteins (BMP), platelet derived growth factor (PDGF), hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α or β-TGF), fibroblastic growth factors, epidermal growth factor (EGF), vascular endothelium growth factor (VEGF), nerve growth factor (NGF) and muscle morphogenic factor (MMP).

The techniques of the invention may be used to develop tissues of ectodermal, mesodermal, and endodermal origin. In a preferred embodiment, growth factors are selected that will promote differentiation of the ES cells and formation of a predetermined tissue type. For example, addition of TGF-β to ESC seeded on three-dimensional matrices induces formation of extracellular matrix characteristic of cartilage tissue. Both activin A and IGF can induce ES cells to produce proteins characteristic of developing liver. RA can induce hES cells to organize into ectodermal structures similar to neuronal tissue. Exposure of ES cells to bone morphogenetic protein, colony stimulating factors specific to bone, and/or PDGF may promote formation of collagen and other bone ECM proteins. Dexamethasone may induce osteogenic differentiation, and ascorbic acid may promote development of osteoclasts. Adipocyte formation may be stimulated with dexamethasone and insulin, and skeletal muscle cell differentiation may be promoted with 5-azacytidine. High concentrations of PDGF in serum-free media without other growth factors can induce development of smooth muscle cells, and substitution of b-FGF for PDGF can promote cardiac muscle cell formation. We have observed spontaneous differentiation of ESC without the use of growth factors or similar biologically active materials, but one skilled in the art will find that the frequency of a particular cell will likely be higher if the ESC environment is manipulated to promote differentiation along a particular path.

In another embodiment, the ESC are mixed with another cell type before implantation. The cell mixture may be suspended in a carrier such as a culture medium or in a gel as described above. Alternatively, the cells may be co-seeded onto a polymer scaffold or combined with a gel that is absorbed into the scaffold. While cumbersome, it may be desirable to seed one cell type directly onto a scaffold and add the second cell type via a gel. Likewise, mixtures of cells may be encapsulated together or separate capsules of ESC and of other cells may be cultured together. Any ratio of ESC to the other cell type or types may be used. One skilled in the art will recognize that this ratio may be easily optimized for a particular application. Exemplary ratios of ESC to other cells are at least 10% (e.g., 1:9), at least 25%, at least 50% (e.g., 1:1), at least 75%, and at least 90%. Smaller ratios, for example, less than 10%, may also be employed.

Any cell type, including connective tissue cells, nerve cells, muscle cells, organ cells, or other stem cells, for example mesenchymal stem cells, may be combined with the ESC to facilitate differentiation or with partially differentiated ESC to further development of a particular tissue type. For example, endothelial cells may be combined with osteogenic cells derived from ESC to promote the co-production of bone and its vasculature. Other exemplary cells that may be combined with undifferentiated or partially differentiated ESC hematopoietic cells and other cells found in bone marrow.

### EXAMPLES

Materials and Methods

All materials were used as received unless otherwise indicated. The following substrates were used: tissue culture polystyrene 75 cm² and 25 cm² flasks (BD Falcon®) and 24-well plates (Falcon®). The α-minimal essential medium (α-MEM), phosphate buffered saline (PBS), fetal bovine serum (FBS, catalog #10437-028), 0.25% trypsin, non enzymatic cell dissociation solution, and gentamicin were obtained from Invitrogen Co. The penicillin G, bovine serum albumin (BSA), amphotericin B (fungizone), AA, hexamethyldisilazane (HMDS), βgP, and DEX were obtained from Sigma Chemical Company. An alkaline phosphatase (ALP) detection kit was obtained from JAS Diagnostics (Miami, Fla.) and an OCN detection kit was obtained from Diagnostic Systems Laboratories Inc. (Webter, Tex.). Mouse embryonic feeder cells were obtained from Cell Essential (Boston, Mass.).

ES Cell Culture

hESC (line H9, passages 25 to 45) were grown as cell aggregates on an inactivated mouse embryonic feeder layer, as previously described [18]. The hESC were passaged every 4 days using 2 mg/mL type IV collagenase (Invitrogen). Undifferentiated hES cell aggregates were removed from mouse feeders with 2 mg/mL collagenase for 2 h. To obtain a single cell suspension, cells were incubated at 37° C. for 5 min in a solution with 1:2 (vol:vol) trypsin to cell dissociation solution with gentle pipetting. Cells were plated at a concentration of 10⁵ cells per cm² in α-MEM supplemented with 10% fetal bovine serum and antibiotics consisting of 167 U/ml penicillin G, 50 μg/ml gentamycin, and 0.3 μg/ml amphotericin B. To examine the potential of the hES cells to produce mineralized extracellular matrix, two differentiation protocols were examined (FIG. 1). The first protocol (FIG. 1A) involved inducing the formation of EB by transferring the hESC aggregates to low attachment plates containing knockout-Dulbecco's modified Eagle's medium supplemented with 20% knockout-serum, 1 mM L-glutamine, 0.1 mM β-mercaptoethanol and 1% nonessential amino acid stock (all from Invitrogen). EB were cultured at 37° C. and 5% CO₂ in a humidified atmosphere, with a change of media on the second day. After 5 days the a single cell suspension was generated and subsequently plated on tissue culture polystyrene. The second protocol (FIG. 1B) involved direct plating of the hESC through skipping the embryoid body step. We were able to obtain significant numbers of bone nodules from hESC without the use of EB and without plating the aggregates (FIGS. 1C,D,E). However, because few of the plated cells attached, undifferentiated hESC aggregates were initially plated on tissue culture dishes for 1 day prior to separating the cells into a single cell suspension.

Osteogenic Differentiation

To stimulate differentiation into osteogenic cells, media containing A-MEM and FBS was supplemented with 50 μg/ml AA, 5 mM βgP and 10⁻⁸M DEX together with antibiotics and fungizone. Through interacting with specific glucocorticoid receptors, DEX has been demonstrated to stimulate osteogenic differentiation for progenitor cells derived from multiple tissues. AA participates in collagen assembly and βgP in mineralizations. The medium was changed every 2-3 days and mineralized areas were observed by light microscopy and by electron microscopy as previously described [19]. Cultures were treated either with or without osteogenic supplements to assess directed or spontaneous differentiation into osteogenic cells, respectively. In some circumstances, individual components of the osteogenic media were employed to examine which components were responsible for the observed response. Unless otherwise stated, this media is referred to as containing osteogenic supplements.

Electron Microscopy

Prior to fixation, culture substrates were washed three times in PBS. Fixation was carried out for a minimum of 2 h in Kamovsky's fixative at 4° C. After rinsing with cacodylate buffer 3 times, the dishes were dehydrated in graded alcohols (50%, 70%, 80% 90%, 95% and 100%) and then air dried in HMDS as previously reported [20]. Overlying cell layers and the collagenous matrix were partially removed with compressed air to facilitate examination of the elaborated extracellular matrix. The samples were then sputter-coated with carbon (≈250A) and examined on a JEOL JSM-5910 scanning electron microscope equipped with a Rontec energy dispersive x-ray (EDX) detector for elemental analysis and mapping. Calcium to phosphate ratios (Ca:P) were obtained by integrating the area under the Ca and P peaks.

Histochemical Analysis and Quantification of Bone Nodules

Alkaline Phosphatase/Von Kossa/Tetracycline Staining

Cell culture plates were fixed in 10% formalin-buffered saline for 20-30 min, washed once with ddH₂O and then left in ddH₂O for 15 min. Plates were then stained for ALP by incubating for 40 min in a solution containing red violet (5-chloro-4-benzamido-2-methylbenzenediazonium chloride hemi(zinc chloride) salt as previously reported [21]. Plates were then rinsed 3-4× in ddH₂O, and stained with 2.5% silver nitrate for 30 min. After rinsing 3-4× in ddH₂O, plates were incubated in sodium carbonate formaldehyde for 1-2 min, rinsed, air dried, and examined by light microscopy. For tetracycline staining, tetracycline was added to the culture media 48 h prior to terminating the cultures and visualized using a fluorescent light box equipped with a digital camera. Bone nodules were identified by the co-localization of ALP and von Kossa staining [22]. Bone nodules within at least three entire wells of six-well plates were quantified under light microscopy for the experimental group. Cultures were also stained with safranin-O/fast green for identification of glycosaminoglycans (cartilage). To compare the frequency of osteogenic cells derived from hESC using both culture methods, the number of mineralized regions that stained positively for both ALP and von Kossa were manually quantified using light microscopic images. The values obtained were normalized to the number of adherent cells at the time of cell plating.

FTIR

FTIR studies were conducted with a Nicolet Magna-IR 500 spectrophotometer. Dry samples were powdered, mixed with KBr, and pressed into pellets. The FTIR spectra were obtained by recording 128 scans between 4000 and 400 cm⁻¹ with a resolution of 4 cm⁻¹. Plots were baseline corrected and analyzed over the range of 900-1725 as previously reported [9]. The mineral-to-matrix ratio was obtained by integrating the area under the curve between 900-1200 cm⁻¹ and dividing by the area under the curve between 1585 and 1725 cm⁻¹. Spectra were also acquired from human humerus (Massachusetts General Hospital Bone Bank) and from hydroxyapatite (Clarkson Chromatography Ltd).

Immunocytochemistry and Flow Cytometry

Cell cultures were fixed in a 4% solution of paraformaldehyde in PBS for 20 min, washed in PBS, and incubated in 0.2% (vol/vol) Triton X-100 with DAPI for 30 min to permeabilize cell membranes and stain the nuclei. After washing in PBS, cells were incubated in 1% (vol/vol) BSA for 20 min, rinsed and then incubated for 1-4 hours with primary antibodies for OCN(R&D Systems) and OCT-4 (BioVision Inc.) at a dilution of 1/100 contained within a 1% (vol/vol) solution of BSA. Cells were then rinsed and stained with the respective secondary antibodies in BSA for 2 h. Any signal greater than that observed with the respective isotype (negative) controls was taken to be positive. As a negative control for immunocytochemistry, human umbilical vein endothelial cells (HUVECs) were used. These cells were cultured in EGM-2 medium (Clonetics, Cambrex BioScience Baltimore, Inc.) with media changes every other day. For flow cytometry, hESC derived without EB after 20 and 30 days in culture (n=1) were stained with propidium iodide (PI) (2 mg/mL) and an ALP monoclonal antibody (B4-78 hybridoma, Developmental Studies Hybridoma Bank, University of Iowa) at a dilution of 1/10 and subsequently analyzed under a fluorescence activated cell sorting (FACS) scan flow cytometer (BD Biosciences). Live cells were analyzed using the Cell Quest software.

Statistical Analysis

Unless otherwise stated, all experiments were performed in triplicate and the data presented are representative of 3 independent experiments. For single comparisons, an unpaired student t-test was used. For multiple comparisons, analysis of variance was performed with the Tukey's honestly significant difference (HSD) test at significance levels of 95%. Error bars in bar graphs represent the standard deviation.

Results

hESC grown on mouse feeders displayed a typical aggregate morphology (FIG. 2A) and stained positively for OCT-4 (FIG. 2B) and ALP (FIG. 2C), both markers for undifferentiated ESC [23]. hESC aggregates on mouse feeders did not stain positive for von Kossa (FIG. 2C) or OCN immunostaining (not shown) indicating the absence of mineralized regions. Collagenase IV treatment removed most of the hESC aggregates leaving the mouse feeders attached to the culture dish (FIG. 2D). For cells cultured with an embryoid body step, placement of cell aggregates into low attachment plates provided a suitable environment for the formation of EB which were held in suspension for 5 days (not shown). When the embryoid body step was omitted, very few of the day 4 hESC obtained from the mouse feeders attached to the culture dish, irrespective of the substrate (FN, gelatin, tissue culture). To circumvent this, after treatment with collagenase IV we plated entire cell aggregates on tissue culture dishes to which cells quickly attached. After 24 h, cells had migrated from the aggregate as observed from light microscopy (FIG. 2E). When hESC were obtained from EB, 1.42±0.33% of the seeded cells attached to the underlying culture dish compared to 2.1±0.60% for the cells that were not obtained from EB. The majority of the cells that did not attach were alive as determined by toluidine blue exclusion and gentle centrifugation of the culture dish (500 g) did not improve the efficiency of cell attachment.

Culturing hESC Without the Embryoid Body Step Affects the Kinetics of Alkaline Phosphatase and Osteocalcin Expression

Undifferentiated hESC exhibited a strong signal for ALP, which is an enzyme expressed by both hESC [24] and osteoblasts [25], amongst other cell types. ALP expression remained relatively constant (p=0.420, n=3) after suspending cell aggregates as EB for 5 days (FIG. 3A). After plating a single cell suspension from the EB, a rapid decrease in ALP was observed in cells treated with growth media, or media containing supplements (FIG. 3A). After 10 days, only the cells cultured in the presence of osteogenic supplements began to re-express significant levels of ALP. The expression of ALP reached a plateau after 20 days. At 27 days, the ALP signal was 10.1 fold (p=0.023, n=3) higher when osteogenic supplements were added to the media. In contrast, for the cells cultured without EB (FIG. 3B), after an initial 58% increase (p=0.052, n=3) in ALP for the group without supplements, the ALP signal for both groups with and without supplements gradually decreased before reaching a plateau slightly above zero. This corresponded well with the flow cytometry data which showed that approximately 10% of the hESC cultured without EB in the presence of osteogenic supplements expressed ALP after both 20 and 30 days (not shown).

OCN, a late marker of osteogenesis that corresponds with induction of mineralization [26], was first detected after 25 days in osteogenic cultures derived from EB containing osteogenic supplements (FIG. 3C) and displayed an upward trend when the cultures were terminated. After 35 days, cultures derived from EB treated with osteogenic supplements displayed a 22.9 fold higher OCN signal (p<0.001, n=3) than those cultures without supplements. In contrast, OCN was first detected after 15 days in cells cultured without EB (FIG. 3D) and quickly reached a plateau with no significant differences observed between the levels of OCN regardless of the supplementation regime.

Culturing hESC Without the Formation of Embryoid Bodies Generates Spontaneous Bone Nodules and Increases Osteogenic Cell Numbers.

After 10-12 days, the cells and ECM produced by cultures grown without EB stained for both ALP and von Kossa, respectively, in both the presence and absence of supplements (FIG. 4A). 3D hemispherical structures appeared (FIG. 4B) that displaced the mineralized regions to the margins of these structures (black arrow). The mineralized nodules did not stain red for safranin 0 (FIG. 4C) demonstrating that glycosaminoglycans were not present in this matrix. The emergence of 3-D structures was evidenced by light microscopy (FIG. 5A) and corresponded with a decrease in ALP activity as observed by qualitative ALP staining.

Visual Observations of ALP Staining Corresponded Well with Biochemical and Calorimetric Analysis.

hESC from EB contained areas of intense ALP staining (FIG. 5A) only when cultured in the presence of DEX. These regions began to mineralize after 4 weeks and stained positively for von Kossa (FIG. 5A), OCN (FIG. 5A), and tetracycline (not shown). However, when cultured in the presence of only growth media or growth media containing AA and βgP, intense ALP staining and mineralized areas were not observed. Addition of serum to the growth medium did not result in an improvement either in the number of cells that adhered or the frequency of bone nodules observed. In contrast, hESC that were cultured without EB formed three dimensional structures, which emerged from the underlying monolayer of cells, stained positively for alkaline phosphatase (FIG. 5A), von Kossa (FIG. 5A), tetracycline (not shown), and OCN (FIG. 5A). The formation of these mineralized aggregates was independent of the presence of AA, βgP and DEX. Von Kossa expression was localized to the margins of the 3-D structures and co-localization of tetracycline demonstrated that these regions were actively mineralizing upon termination of the cultures (not shown). To ensure that the data were not false positives, a number of controls were performed. Human umbilical vein endothelial cells which served as a negative control for immunocytochemistry did not stain positive for OCN (not shown). Furthermore, day 4 aggregates of hESC grown on mouse feeders which served as a negative control did not stain positive for von Kossa, tetracycline, or OCN.

To compare the frequency of osteogenic cells derived from hESC, the number of mineralized regions that stained positively for both ALP and von Kossa were quantified after 30 days and 14 days in cultures obtained with and without EB, respectively. Cells grown from EB cultured in the presence of osteogenic supplements produced 5.1±2.4 bone nodules per 10,000 cells, whereas the cells grown from EB without supplements did not produce bone nodules (FIG. 5B). In comparison, cells that were grown in media with and without osteogenic supplements after being plated for one day produced 39.1±17.8 and 13.8±3.1 bone nodules per 10,000 cells, respectively. Cells cultured without EB in the presence of osteogenic supplements produced significantly more bone nodules compared to cells cultured with EB in the presence of osteogenic supplements (P=0.018). In addition, even more bone nodules (78.0+/−19.6 nodules per 10,000 attached cells) were obtained when the hESC were directly plated as a single cell suspension, thus avoiding plating the aggregates for one day.

Differentiated hESC Produce Many of the Hallmarks of de Novo Bone Formation Including a Mineralized Collagenous Matrix Containing Calcium Phosphate.

When hESC were cultured from 5 day EB in the absence of supplements (FIG. 6A), or in the presence of AA and βgP but not DEX (not shown), the matrix produced was reminiscent of unmineralized collagen [19] as observed by SEM. However, regions that appeared to contain ectopically deposited calcium and phosphorus were observed and verified by a high calcium to phosphorus ratio (2.71:1) and standard deviation (0.64) as observed with energy dispersive x-ray analysis (EDX). When DEX was added in addition to the other supplements (FIG. 6B), distinct areas of mineralization were observed within the collagen fibers, which displayed a Ca:P of 1.78:1±0.01. The extracellular matrix produced by the cells obtained from embryoid body free cultures without supplements appeared to contain small mineral deposits and had a Ca:P of 2:00:1±0.10 (FIG. 6C). When osteogenic supplements were added, regions containing thickened fibers and large mineral deposits were observed (FIG. 6D) and the Ca:P decreased to 1.64:1±0.04. Hydroxyapatite samples served as a positive control and displayed a Ca:P of 1.69:1±0.11 which was similar to its theoretical value of 1.67:1. Human humerus (FIG. 6E) contained thick mineralized collagen fibers with a Ca:P of 1.52:1±0.09. The decrease in Ca:P ratios observed when osteogenic supplements were added, regardless of the inclusion of EB, was primarily due to an increase in the presence of phosphorous as observed from the EDX spectra.

Fourier Transform Infra-Red (FTIR) analysis was conducted to examine and compare the mineralized extracellular matrix to hydroxyapatite and human bone (FIG. 6F). The mineral peak from hydroxyapatite and the mineral and matrix peaks from human bone were comparable to the extracellular matrix produced by the hESC irrespective of whether the cells were derived from EB. However, human bone, had mineral to matrix ratio of 4.7:1 (FIG. 6F2) which was substantially higher than for the ECM produced by hESC derived with EB in the absence (0.5:1, FIG. 6F3) and presence (2.1:1, FIG. 6F4) of osteogenic supplements. In contrast to the ECM produced by hESC derived with EB, the ECM produced by hESC derived without EB was similar regardless of whether the cells were cultured in the absence (1.5:1, FIG. 6F3) or presence (1.4:1, FIG. 6F4) of osteogenic supplements.

The hESC culture Protocol Affects the Sequence of de Novo Bone Formation

To examine if the osteogenic cells derived from hESC could produce the cement line matrix, the first matrix produced by differentiating osteogenic cells and separates new bone from the old bone surface during remodeling, electron microscopy was employed. Compressed air was used to remove the overlying cell and collagenous matrix to expose the underlying ECM/culture dish interface. The hESC derived from EB that were cultured in the absence of supplements (FIG. 7A) or in the presence of AA and βgP (not shown) did not produce the cement line matrix. However, when DEX was added in addition to the other osteogenic supplements, a mineralized cement line matrix was revealed as evidenced by SEM (FIGS. 7B,C) and by elemental mapping using EDX (not shown). In contrast, when hESC were derived from an embryoid body free system, cement line matrix on the culture dish was not observed, regardless of the addition of osteogenic supplements (FIGS. 7D,E). However, globules that are reminiscent of the cement line matrix were observed suspended within the overlying collagenous matrix (FIG. 7F).

Discussion

In one embodiment, hESC differentiate into osteogenic cells. Osteogenic cells were identified based in the biomarkers ALP, OCN, bone nodule formation, and based on the formation of cement line matrix. Given that the frequency of osteoprogenitor cells can only be demonstrated retrospectively through examining the culture surface for de novo bone formation [27], we quantified the number of bone nodules as an indirect measure of the number of osteoprogenitors within our cultures. The number of bone nodules produced by hESC derived from EB in our study (5.1±2.4 bone nodules per 10,000 attached cells) was similar to the number reported in previous experiments where hESC were derived using a similar protocol, except that their EBs were cultured in the presence of serum. Specifically, Bielby et al. reported a maximum of 38 bone nodules from differentiated hESC in a 35 mm dish (which has a surface area of 9.6 cm²) at a seeding density 5200 cells/cm² which equates to 7.6 bone nodules per 10,000 cells [12]. Since approximately 85% of their cells attached [28], this translates into 8.9 nodules per 10,000 attached cells. Although it is useful to compare results between studies, these comparisons must be interpreted with caution given that previous experiments with hESC have used 10 mM βgP [11, 12] (twice the concentration used in this study) which has been associated with increased levels of dystrophic mineralization [17]. EB have been used as a model for recapitulating the simultaneous formation of multiple tissues during embryonic development; however, a system devoid of EB may be useful to improve the derivation efficiency of osteogenic cells. As discussed above, a 7.6 fold increase (39.1±17.8) in the number of bone nodules was observed when EB were omitted from the culture protocol. In addition, the nodules produced from cultures without EB formed after 10-12 days compared to after 4 weeks in cultures derived from EB. The relatively rapid production of bone nodules in cultures derived without EB is supported by the early detection of OCN as observed in FIG. 3. Thus, the use of cell culture techniques that do not employ EB may be used to produce cell cultures that exhibit bone nodules sooner, for example, at least 20%, 30%, 40%, 50%, or 60% sooner, than cell culture techniques employing EB.

To engineer human bone tissue in vitro, it is relevant to thoroughly characterize the matrix produced by the cell source of interest and compare it to native bone. Although numerous staining techniques have been used to detect the presence of osteoblasts in culture, expression of osteogenic markers such as ALP does not directly correlate with production of bone nodules [22]. Conventional wisdom holds that cells capable of forming bone are more useful for engineering bone tissue than cells that express osteogenic markers yet do not produce bone. Therefore, in addition to using classic stains to identify osteogenic cells, it is imperative to examine the matrix produced by the cells. Using a typical rodent cell culture system, osteogenesis in vitro has been demonstrated to culminate in the formation of mineralized nodules which are discrete islands of bone that display histological, ultrastructural and immunohistochemical similarities to bone formed in vivo [22, 29]. With respect to ESC, to date there is only one study that has characterized the mineralized matrix produced by a differentiated murine population [9]. They found that the mineralization process did not parallel conventional osteogenesis and their spectroscopic analysis demonstrated that the calcium-to-phosphorus ratio (Ca:P) of the mineral phase was 1.26:1 compared to 1.67:1 for hydroxyapatite. Therefore, it is unclear whether the nodule-like structures described thus far in ESC cultures are indeed bone nodules that resemble bone formed in situ or are representative of dystrophic mineralization. Given that von Kossa and/or alizarin red are the primary stains used for identification and quantification of in vitro bone nodules from osteogenic cultures of h(ES) cells [11, 12], the positive identification of in vitro bone formed from hESC has yet to verified. We demonstrated that differentiated hESC can produce a mineralized matrix that displays colocalized staining for ALP and von Kossa and displays many of the hallmarks of de novo bone formation including a cement line matrix and mineralized collagen. We show that the matrix produced by the differentiated hESC, irrespective of the culture protocol, contains an apatitic mineral phase with calcium and phosphorous in a ratio that is similar to that for hydroxyapatite and human bone. Although hypertrophic chondrocytes may express alkaline phosphatase and osteocalcin, and produce a mineralized collagenous matrix that stains positively for von Kossa [30, 31], the absence of glycosaminoglycans and the presence of cement line matrix demonstrates that that these cells are osteogenic.

The results reported previously for murine ESC cultured without EB are in contrast to the results we present here and likely represent innate differences between humans and mice. For example, murine ESC cultured without the embryoid body step fail to spontaneously differentiate into the osteogenic cells [9] and, ALP expression remains relatively constant, regardless of the presence of osteogenic supplements. Therefore, for clinical application of osteogenic cells derived from ESC, it remains crucial to focus efforts on understanding the differentiation processes in the human system. Differences between murine and human ESC have been previously described in detail elsewhere [14, 32].

While omitting EB from the culture protocol improves the efficiency of osteogenic differentiation, the emergence of 3-D structures impedes the development of in vitro bone. Therefore, purification of the osteogenic population for bone engineering applications would likely need to be performed prior to the emergence and dominance of these structures at day 10-12. This is when the hESC have lost their stem cell properties as evidenced by a decrease in ALP. Unlike cultures of hESC that were derived from EB where ALP expression re-emerged, corresponding to the appearance of osteogenic cells, for the hESC derived without EB, the ALP signal continued to decrease before reaching a low, yet detectable, level. Without being bound by any particular theory, this low level of ALP and the relatively low OCN signal which quickly reached a plateau was likely produced from the osteogenic cells which were displaced to the margins of the 3-D structures. Although speculative, we believe that the emergence of the 3-D structures corresponded to the appearance and dominance of a non-osteogenic cell type. The presence of mineralized globules of cement line within the collagenous matrix suspended above the culture surface was likely due to the forces exerted by these cells which displaced the bone nodules to the margins of the 3-D structures. In addition, the low mineral to matrix ratio observed for hESC cultured without EB compared to the matrix produced by hESC cultured with EB may be explained by the dominance of a non-osteogenic cell type which hindered the growth and maturation of the bone matrix.

### REFERENCES

[1] Langer, R.; Vacanti, J. P. Science 1993, 260, 920-926.
[2] Alsberg, E.; Anderson, K. W.; Albeiruti, A., et al. J Dent Res 2001, 80, 2025-2029.
[3] Bhatia, M. Ann N Y Acad Sci 2005, 1044, 24-28.
[4] Thomson, J. A.; Itskovitz-Eldor, J.; Shapiro, S. S., et al. Science 1998, 282, 1145-1147.
[5] Perlingeiro, R. C.; Kyba, M.; Daley, G. Q. Development 2001, 128, 4597-4604.
[6] zur Nieden, N. I.; Kempka, G.; Ahr, H. J. Differentiation 2003, 71, 18-27.
[7] Bourne, S.; Polak, J. M.; Hughes, S. P., et al. Tissue Eng 2004, 10, 796-806.
[8] Buttery, L. D.; Bourne, S.; Xynos, J. D., et al. Tissue Eng 2001, 7, 89-99.
[9] Shimko, D. A.; Burks, C. A.; Dee, K. C., et al. Tissue Eng 2004, 10, 1386-1398.
[10] Bronson, S. K. Methods Enzymol 2003, 365, 241-251.
[11] Sottile, V.; Thomson, A.; McWhir, J. Cloning Stem Cells 2003, 5, 149-155.
[12] Bielby, R. C.; Boccaccini, A. R.; Polak, J. M., et al. Tissue Eng 2004, 10, 1518-1525.
[13] Cao, T.; Heng, B. C.; Ye, C. P., et al. Tissue Cell 2005, 37, 325-334.
[14] Ginis, I.; Luo, Y.; Miura, T., et al. Dev Biol 2004, 269, 360-380.
[15] Itskovitz-Eldor, J.; Schuldiner, M.; Karsenti, D., et al. Mol Med 2000, 6, 88-95.
[16] Komori, T.; Yagi, H.; Nomura, S., et al. Cell 1997, 89, 755-764.
[17] Bonewald, L. F.; Harris, S. E.; Rosser, J., et al. Calcif Tissue Int 2003, 72, 537-547.
[18] Amit, M.; Carpenter, M. K.; Inokuma, M. S., et al. Dev Biol 2000, 227, 271-278.
[19] Karp, J. M.; Shoichet, M. S.; Davies, J. E. J Biomed Mater Res 2003, 64A, 388-396.
[20] Slizova, D.; Krs, O.; Pospisilova, B. J Endovasc Ther 2003, 10, 285-287.
[21] Purpura, K. A.; Aubin, J. E.; Zandstra, P. W. Biotechniques 2003, 34, 1188-+.
[22] Purpura, K. A.; Aubin, J. E.; Zandstra, P. W. Stem Cells 2004, 22, 39-50.
[23] Klimanskaya, I.; Chung, Y.; Meisner, L., et al. Lancet 2005, 365, 1636-1641.
[24] Draper, J. S.; Pigott, C.; Thomson, J. A., et al. J Anat 2002, 200, 249-258.
[25] Aubin, J. E.; Liu, F.; Malaval, L., et al. Bone 1995, 17, 77S-83S.
[26] Aubin, J. E. J Cell Biochem Suppl 1998, 30-31, 73-82.
[27] Purpura, K. A.; Aubin, J. E.; Zandstra, P. W. Biotechniques 2003, 34, 1188-1192, 1194, 1196 passim.
[28] Bielby, R. C., Pryce R S, Hench L L, Polak J M., Tissue Eng. 2005 Mar-Apr; 11(3-4):479-88.
[29] Davies, J. E. Anatomical Record 1996, 245, 426-445.
[30] Schmitt, B.; Ringe, J.; Haupl, T., et al. Differentiation 2003, 71, 567-577.
[31] Lian, J. B.; McKee, M. D.; Todd, A. M., et al. J Cell Biochem 1993, 52, 206-219.
[32] Wei, C. L.; Miura, T.; Robson, P., et al. Stem Cells 2005, 23, 166-185.
[33] Heng, B. C.; Cao, T.; Stanton, L. W., et al. J Bone Miner Res 2004, 19, 1379-1394.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method of culturing embryonic stem cells, comprising:
providing a plurality of embryonic stem cells (ESC);
seeding the ESC on a substrate or suspending them in a culture medium as a single cell culture; and
incubating the seeded or suspended ESC under conditions in which they can differentiate to obtain a population of cells, wherein
the ESC are not stimulated to form embryoid bodies, and
wherein the proportion of a predetermined differentiated cell type in the population of cells is at least 2 times greater than the proportion of the predetermined differentiated cell type in a population obtained in the same manner except that the ESC are stimulated to form embryoid bodies.

2. The method of claim 1, wherein the proportion is at least 3 times greater.

3. The method of claim 1, wherein the proportion is at least 4 times greater.

4. The method of claim 1, wherein the proportion is at least 5 times greater.

5. The method of claim 1, wherein the proportion is at least 6 times greater.

6. The method of claim 1, wherein the proportion is at least 7 times greater.

7. The method of claim 1, wherein providing comprises culturing a population of ESC over a feeder layer.

8. The method of claim 7, further comprising separating the cells into individual cells.

9. The method of claim 1, wherein the plurality of embryonic stem cells comprising aggregates of cells, and wherein these aggregates are seeded.

10. The method of claim 1, wherein the plurality of embryonic stem cells comprising aggregates of cells, and wherein providing comprises separating the aggregates into smaller aggregates, and wherein at least a portion of the smaller aggregates are seeded.

11. The method of claim 1, wherein providing comprises separating undifferentiated ESC into single cells.

12. The method of claim 1, wherein providing comprises separating OCT-4/alkaline phosphatase positive ESC into single cells.

13. The method of claim 1, wherein providing does not comprise disposing the ESC over a feeder layer.

14. The method of claim 1, wherein seeding comprises seeding the cells on a two-dimensional substrate or a three-dimensional substrate.

15. The method of claim 1, wherein suspending comprises encapsulating the ESC in a hydrogel and placing the encapsulated ESC in a culture medium.

16. The method of claim 1, wherein suspending comprises placing the ESC in a bioreactor and wherein incubating occurs in the bioreactor.

17. The method of claim 1, wherein the ESC are human ESC.

18. The method of claim 1, further comprising culturing the seeded or suspended cells in the presence of at least one growth factor or cytokine.

19. The method of claim 18, wherein the at least one growth factor or cytokine is selected from dexamethasone, leptin, sortilin, transglutaminase, prostaglandin E, 1,25-dihydroxyvitamin D3, ascorbic acid, β-glycerophosphate, TAK-778, statins, interleukins such as IL-3 and IL-6, growth hormone, steel factor (SF), activin A (ACT), retinoic acid (RA), epidermal growth factor, bone morphogenetic proteins (BMP), platelet derived growth factor (PDGF), hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α or β-TGF), fibroblastic growth factors, epidermal growth factor (EGF), vascular endothelium growth factor (VEGF), nerve growth factor (NGF) and muscle morphogenic factor (MMP).

20. The method of claim 19, wherein seeded ESC are cultured in the presence of dexamethasone, ascorbic acid, and β-glycerophosphate.

21. The method of claim 18, wherein the growth factor is TGF-beta and the predetermined cell type produces cartilage tissue.

22. The method of claim 18, wherein the growth factor is selected from activin A and IGF and the predetermined cell type is liver progentor cells.

23. The method of claim 18, wherein the growth factor is retinoic acid and the predetermined cell type is neuronal cells.

24. The method of claim 18, wherein the growth factor is selected from bone morphogenetic protein, colony stimulating factor, and PDGF and the predetermined cell type produces collagen.

25. The method of claim 18, wherein the at least one growth factor is dexamethasone and insulin and the predetermined cell type is adipocytes.

26. The method of claim 18, wherein the growth factor is 5-azacytidine and the predetermined cell type is skeletal muscle cells.

27. The method of claim 18, wherein the growth factor is PDGF and the predetermined cell type is smooth muscle cells.

28. The method of claim 18, wherein the growth factor is b-FGF and the predetermined cell type is cardiac muscle cells.

29. The method of claim 18, wherein the growth factor is ascorbic acid and the predetermined cell type is osteoclasts.

30. A single cell population of undifferentiated embryonic stem cells (ESC) in contact with a growth factor or cytokine.

31. The single cell population of claim 30, wherein the predetermined lineage is mesodermal.

32. The single cell population of claim 30, wherein the predetermined lineage is osteogenic.

33. The single cell population of claim 30, wherein the ESC are hESC.

34. The single cell population of claim 33, wherein at least 90% of the cells express one or more of OCT-4, ALP, nanog, TRA-1-60, and TRA-1-81 and do not express SSEA-1.

35. The single cell population of claim 30, wherein the ESC are mESC.

36. The single cell population of claim 35, wherein express one or more of OCT-4, ALP, nanog, and SSEA-1 and do not express SSEA-3 or SSEA-4.

37. The single cell population of claim 30, wherein the population is capable of differentiating along a predetermined lineage with a frequency that is at least two times greater than the frequency for a population of human embryonic stem cells recovered from embryoid bodies.

38. The single cell population of claim 30, wherein the population is capable of differentiating to express a predetermined phenotype within an elapsed time (“the first elapsed time”) that is less than an elapsed time (“the second elapsed time”) before expression of the phenotype by a population of cells that are stimulated to form embryoid bodies.

39. The single cell population of claim 30, wherein the at least one growth factor or cytokine is selected from dexamethasone, leptin, sortilin, transglutaminase, prostaglandin E, 1,25-dihydroxyvitamin D3, ascorbic acid, β-glycerophosphate, TAK-778, statins, interleukins such as IL-3 and IL-6, growth hormone, steel factor (SF), activin A (ACT), retinoic acid (RA), epidermal growth factor, bone morphogenetic proteins (BMP), platelet derived growth factor (PDGF), hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α or β-TGF), fibroblastic growth factors, epidermal growth factor (EGF), vascular endothelium growth factor (VEGF), nerve growth factor (NGF) and muscle morphogenic factor (MMP).

40. A method of culturing embryonic stem cells, comprising:
providing a plurality of embryonic stem cells (ESC);
seeding the ESC on a substrate or suspending them in a culture medium as a single cell culture; and
incubating the seeded or suspended ESC under conditions in which they can differentiate to obtain a population of cells, wherein
the ESC are not stimulated to form embryoid bodies, and
wherein at least a portion of the cells express a predetermined phenotype during the step of incubating, and wherein the elapsed time (“the first elapsed time”) before the predetermined phenotype is expressed is less than an elapsed time (“the second elapsed time”) for a population of cells obtained in the same manner except that the ESC are stimulated to form embryoid bodies.

41. The method of claim 40, wherein the first elapsed time is at least 20% less than the second elapsed time.

42. The method of claim 40, wherein the first elapsed time is at least 30% less than the second elapsed time.

43. The method of claim 40, wherein the first elapsed time is at least 40% less than the second elapsed time.

44. The method of claim 40, wherein the first elapsed time is at least 50% less than the second elapsed time.

45. The method of claim 40, wherein the first elapsed time is at least 60% less than the second elapsed time.

46. The method of claim 40, wherein providing comprises culturing a population of ESC over a feeder layer.

47. The method of claim 40, further comprising separating the cells into individual cells.

48. The method of claim 40, wherein the plurality of embryonic stem cells comprising aggregates of cells, and wherein these aggregates are seeded.

49. The method of claim 40, wherein the plurality of embryonic stem cells comprising aggregates of cells, and wherein providing comprises separating the aggregates into smaller aggregates, and wherein at least a portion of the smaller aggregates are seeded.

50. The method of claim 40, wherein providing comprises separating ac4-alkaline phosphatase positive ESC into single cells.

51. The method of claim 40, wherein providing does not comprise disposing the ESC over a feeder layer.

52. The method of claim 40, wherein seeding comprises seeding the cells on a two-dimensional substrate or a three-dimensional substrate.

53. The method of claim 40, wherein suspending comprises encapsulating the ESC in a hydrogel and placing the encapsulated ESC in a culture medium.

54. The method of claim 40, wherein suspending comprises placing the ESC in a bioreactor and wherein incubating occurs in the bioreactor.

55. The method of claim 40, wherein the ESC are human ESC.

56. The method of claim 40, further comprising culturing the seeded or suspended cells in the presence of at least one growth factor or cytokine.

57. The method of claim 56, wherein the at least one growth factor or cytokine is selected from dexamethasone, leptin, sortilin, transglutaminase, prostaglandin E, 1,25-dihydroxyvitamin D3, ascorbic acid, β-glycerophosphate, TAK-778, statins, interleukins such as IL-3 and IL-6, growth hormone, steel factor (SF), activin A (ACT), retinoic acid (RA), epidermal growth factor, bone morphogenetic proteins (BMP), platelet derived growth factor (PDGF), hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α or β-TGF), fibroblastic growth factors, epidermal growth factor (EGF), vascular endothelium growth factor (VEGF), nerve growth factor (NGF) and muscle morphogenic factor (MMP).

58. The method of claim 56, wherein seeded ESC are cultured in the presence of dexamethasone, ascorbic acid, and β-glycerophosphate.

59. The method of claim 56, wherein the growth factor is TGF-beta and the predetermined phenotype is production of cartilage tissue.

60. The method of claim 56, wherein the growth factor is selected from activin A and IGF and the predetermined phenotype is production of proteins characteristic of developing liver.

61. The method of claim 56, wherein the growth factor is retinoic acid and the predetermined phenotype is production of ectodermal structures.

62. The method of claim 56, wherein the growth factor is selected from bone morphogenetic protein, colony stimulating factor, and PDGF and the predetermined phenotype is production of at least bone extracellular matrix component.

63. The method of claim 56, wherein the at least one growth factor is dexamethasone and insulin and the predetermined phenotype is characteristic of adipocytes.

64. The method of claim 56, wherein the growth factor is 5-azacytidine and the predetermined phenotype is characteristic of skeletal muscle cells.

65. The method of claim 56, wherein the growth factor is PDGF and the predetermined cell type is characteristic of smooth muscle cells.

66. The method of claim 56, wherein the growth factor is b-FGF and the predetermined cell type is characteristic of cardiac muscle cells.

67. The method of claim 18, wherein the growth factor is ascorbic acid and the predetermined cell type is osteoclasts.

68. A method of obtaining a population of osteogenic cells, comprising:
providing a population of embryonic stem cells (ESC);
seeding the ESC on a substrate as a single cell culture; and
incubating the seeded ESC in the presence of an osteogenic differentiation factor, wherein
the ESC are not stimulated to form embryoid bodies.

69. The method of claim 68, wherein the osteogenic differentiation factor is dexamethasone.

70. The method of claim 68, wherein incubating further comprises incubating the seeded ESC in the presence of ascorbic acid and beta-glycerophosphate.

71. The method of claim 68, wherein the proportion of osteogenic cells in the incubated ESC is at least 2 times greater than the proportion of osteogenic cells in a population of cells obtained in the same manner except that the ESC are stimulated to form embryoid bodies.

72. The method of claim 71, wherein the proportion is at least 3 times greater.

73. The method of claim 71, wherein the proportion is at least 4 times greater.

74. The method of claim 71, wherein the proportion is at least 5 times greater.

75. The method of claim 71, wherein the proportion is at least 6 times greater.

76. The method of claim 71, wherein the proportion is at least 7 times greater.

77. The method of claim 68, wherein the ESC are human ESC.

78. The method of claim 68, wherein the seeded ESC produce bone nodules during the step of incubating, and wherein the elapsed time (“the first elapsed time”) before the bone nodules are produced is less than an elapsed time (“the second elapsed time”) for a population of cells obtained in the same manner except that the ESC are stimulated to form embryoid bodies.

79. The method of claim 78, wherein the first elapsed time is at least 20% less than the second elapsed time.

80. The method of claim 78, wherein the first elapsed time is at least 30% less than the second elapsed time.

81. The method of claim 78, wherein the first elapsed time is at least 40% less than the second elapsed time.

82. The method of claim 78, wherein the first elapsed time is at least 50% less than the second elapsed time.

83. The method of claim 78, wherein the first elapsed time is at least 60% less than the second elapsed time.
